(19) United States
(12) Patent Application Publication
(10) Pub. No.: US 2013/0230586 A1
(43) Pub. Date: Sep. 5, 2013

(54) Treatment of Tinnitus and Related Auditory Dysfunctions

(71) Applicants: Clauda Couto De Barros Coelho, Taquari RS (BR); Ricardo Rodrigues Figueiredo, Volta Redonda/RJ (BR); Dirk De Ridder, Zelzate (BE); Matteo De Nora, Monaco (MC); Berthold Langguth, Regenburg (DE); Ana Belen Elgoyhen, Buenos Aires (AR)

(72) Inventors: Clauda Couto De Barros Coelho, Taquari RS (BR); Ricardo Rodrigues Figueiredo, Volta Redonda/RJ (BR); Dirk De Ridder, Zelzate (BE); Matteo De Nora, Monaco (MC); Berthold Langguth, Regenburg (DE); Ana Belen Elgoyhen, Buenos Aires (AR)

(73) Assignee: LINK RESEARCH & GRANTS CORPORATION, Principality of Monaco (MC)

(21) Appl. No.: 13/877,370

(22) PCT Filed: Oct. 2, 2010
(86) PCT No.: PCT/IB2010/054456

§ 371 Date: May 21, 2013

(51) Int. Cl. A61K 9/00

(52) U.S. Cl. CPC A61K 9/0002
USPC 424/452; 424/465; 514/654

(57) ABSTRACT

The invention provides an extended-release dosage form of cyclobenzaprine for use in the treatment of tinnitus and related auditory dysfunctions by once-a-day oral administration, wherein the dosage form is a tablet or capsule comprising cyclobenzaprine as active agent in an amount from 10-80 mg, preferably from 10-60 mg. The active agent is associated with a polymer coating or matrix that comprises a water-insoluble polymer, the polymer coating or matrix providing the dosage form with an extended release of the active agent over at least 12 hours and preferably over at least 16 hours when the dosage form is administered to a patient.

### FIELD OF THE INVENTION

This invention relates to the treatment of tinnitus and related common auditory dysfunctions such as hyperacusis, auditory hallucinations, misophonia, phonophobia and central auditory processing disorders.

### BACKGROUND OF THE INVENTION

### Tinnitus

Tinnitus is the phantom sensation of hearing in the absence of external sounds. Two main types can be identified: (1) objective tinnitus, which is caused by sounds generated somewhere in the body; (2) subjective tinnitus, which is the perception of meaningless sounds without any physical sound being present. Objective tinnitus is rare and is caused by a sound in the body, such as turbulent flow of blood or muscle contractions in the head. Such tinnitus can be heard by an observer in contrast to subjective tinnitus, which can only be heard by the individual who has the tinnitus. Subjective tinnitus is the most prevalent type of tinnitus. Tinnitus sounds can take a variety of forms such as buzzing, ringing, whistling, hissing or a range of other sounds. For some people it can even sound like music or singing. It can be a benign sound or it can prevent its sufferers from sleep or the ability to do intellectual work. All degrees of subjective tinnitus occur in between these extremes. Tinnitus is also related to other symptoms, such as hyperacusis, auditory hallucinations, misophonia, phonophobia and central auditory processing disorders. Affective disorders, such as anxiety and depression, often accompany severe tinnitus and that form of tinnitus can lead to suicide. Tinnitus is most likely related to altered neuronal activity which leads to plastic changes in the central auditory pathway derived from a distorted input. However, the mechanisms underlying the different forms of tinnitus remain incompletely understood.

Tinnitus often occurs as a result of dysfunction of the hearing system, such as from noise exposure, presbyacusis or from administration of specific pharmacologic agents. It can also be caused as the result of ear or head injuries, some diseases of the ear, ear infections and emotional stress. Perhaps the most common source of chronic tinnitus is exposure to loud sound. The noise causes permanent damage to the sound-sensitive cells of the cochlea, a spiral shaped organ in the inner ear. Carpenters, pilots, soldiers, rock musicians, street repair-workers are among those people whose jobs put them at risk. But also recreational use of sound, like MP3 players at maximal volume can produce damage. In addition, a long list of drugs can induce tinnitus. In some cases the causative agent remains unknown. One in 10 adults have clinically significant tinnitus (regular prolonged spontaneous tinnitus lasting 5 minutes or more), and for 1 in 100 adults tinnitus severely affects their ability to lead a normal life. Estimates indicate that 13 million people in western Europe and the USA currently seek medical advice for their tinnitus. Over 4 million prescriptions are written each year for tinnitus relief but these are all for off-label drugs from a wide variety of therapeutic classes and most are associated with considerable side effects. Despite the significant unmet clinical need for a safe and effective drug targeting tinnitus relief, there is currently no FDA-approved drug on the market that targets tinnitus.

Tinnitus can be associated with common auditory dysfunctions such as hyperacusis, distortion of sounds, misophonia, phonophobia and central auditory processing disorders. An extremely rare condition called “exploding head syndrome” has sometimes been called “explosive tinnitus”; however exploding head syndrome is not encompassed within the normal meaning of tinnitus or associated common auditory dysfunctions, and the present invention does not cover the treatment of exploding head syndrome.

There are several therapeutic approaches to alleviate tinnitus, however, they have limited results and most patients are left unsatisfied. This includes: 1. counselling which can help the patient to cope with his tinnitus; 2. if tinnitus is accompanied by hearing loss, a hearing aid can help with tinnitus management; 3. sound therapy, also known as sound enrichment; 4. Tinnitus Retraining Therapy, a combination of counseling and sound therapy; 5. cognitive-behavioral therapy; 6. repetitive transcranial magnetic stimulation; 7. epidural cortical stimulation with implantable electrodes and 8. a series of off-label drugs like antidepressants, anxiolytics, anesthetics, anticonvulsants, analgesics, antiarrythmics, herbal medicines, anticoagulants, sedative-hypnotics, antihistaminergic compounds, antipsychotics, antioxidants, vasodilators, among others.

Specifically, the following proposals have been made in the patent literature, but have not been proven for widespread use:

U.S. Pat. No. 4,735,968 discloses a method of treating tinnitus with aminoxyacetic acid (ADAA) administered orally. U.S. Pat. No. 4,954,486 proposed treating tinnitus symptoms with furosemide. U.S. Pat. No. 5,668,117 proposed treatment of tinnitus with a carbonyl trapping agent in combination with antidepressants or antianxiety medications; anti-convulsants; lidocaine; aminooxyacetic acid; praxilene; aniracetam; piracetam; 13-cisretionic acid; and 13-trans-retinoic acid. U.S. Pat. No. 5,716,961 discloses the treatment of tinnitus using specific neuroprotective agents. U.S. Pat. No. 5,863,927 proposed to treat tinnitus with dextromethorphan in combination with a debrisoquin hydroxylase inhibitor. U.S. Pat. No. 6,358,540 disclosed the treatment of tinnitus with an herbal composition. U.S. Pat. Nos. 6,656,172 and 6,969,383 proposed treatment of tinnitus using a catheter to infuse a therapeutic agent for example an agent comprising a local anesthetic such as lidocaine, or a GABA agonist. U.S. Pat. No. 6,713,490 discloses a compound which is (R)-6[2-[4-(3-fluorophenyl)-4-hydroxy-1-piperidibyl]-1-hydroxyethyl]-3,4-dihydro-2(1H)-quinolinone inter alia for the treatment of tinnitus. U.S. Pat. No. 6,770,661 disclosed various aryl substituted pyridines inter alia as antitinnitus agent.

Cyclobenzaprine

Cyclobenzaprine is a skeletal muscle relaxant. The exact mechanism of action for cyclobenzaprine is unknown. Current research appears to indicate that cyclobenzaprine acts on the locus coeruleus where it results in increased norepinephrine release, potentially through the gamma fibers which innervate and inhibit the alpha motor neurons in the ventral horn of the spinal cord. Decreased firing of the alpha motor neuron results in decreased muscular tone. Cyclobenzaprine is a muscle relaxant acting primarily on the central nervous system. It is structurally similar to Amitriptyline, differing by only one double bond. Cyclobenzaprine is typically prescribed to relieve pain and muscle spasms. Typically, muscle spasms occur in an injury to stabilize the affected body part and prevent further damage. Whereas this is beneficial in acute injury, muscle spasm frequently persists over time, becomes dysfunctional and can increase the pain level. It is believed that by decreasing muscular spasm, pain is diminished. A common application would be that of a whiplash injury in a car accident. Cyclobenzaprine has also been studied in the treatment of fibromyalgia. In a study of 120 fibromyalgia patients, those receiving Cyclobenzaprine (10 to 40 mg) over a 12 week period had significantly improved quality of sleep and pain score. Interestingly, there was also a reduction in the total number of tender points and muscle tightness. It is also prescribed off-label as a sleeping-aid.

U.S. Pat. No. 7,387,793 proposes an extended release form of cyclobenzaprine for treating muscle spasm associated with painful musculoskeletal conditions.

Cyclobenzaprine has been proposed in U.S. Pat. No. 6,632,843 and patent application US 2006/06178511 for the treatment of bruxism by topical administration as a cream onto the skin overlying accessible muscles of mastication. Possible accessory alleviation of the other symptoms including tinnitus was also mentioned

GB Patent Specification 1 334 326 proposes a composition having skeletal muscle relaxant activity comprising cyclobenzaprine and aspirin, which is intended to reduce the side effects of aspirin when administered in recommended dosages, such side effects including provoking epigastric distress, nausea, vomiting and tinnitus and more important, gastric erosion, exacerbation of peptic ulcer symptoms, perforation and hemorrhage.

Despite many suggestions for tinnitus treatment discussed above, there remains a need for an effective treatment of tinnitus that could be widely accepted.

As-yet unpublished PCT patent application PCT/IB2010/051373, filed 30 Mar. 2010, relates to cyclobenzaprine for use in the treatment of tinnitus and related auditory dysfunctions by oral administration or by parenteral administration through intramuscular, intravenous, subcutaneous or intrathecal injection or infusion, and presents data demonstrating the efficacy of cyclobenzaprine for these treatments. This unpublished PCT patent application mentions the use of extended release cyclobenzaprine for tinnitus by once-a-day administration but does not provide any details thereupon, neither of the means for providing extended release, nor any other details.

Background Art on Extended-Release Drug Formulations

Considerable efforts have been devoted to developing matrix tablet-based and multi-particulate capsule-based drug delivery systems for oral applications, in order to make therapeutic agents available at a constant rate at or near the absorption site. The resulting delayed absorption of therapeutic agents generally results in desired plasma concentrations leading to maximum efficacy and minimum toxic side effects.

U.S. Pat. No. 4,839,177 concerns a system for the controlled-rate release of active substances consisting of a core comprising an active substance and (a) a polymeric material having a high degree of swelling on contact with water and a gellable polymeric material and/or (b) a single polymeric material having both swelling and gelling properties, and a support platform of a water insoluble polymeric material applied to the core.

U.S. Pat. No. 4,851,228 discloses a multi-particulate osmotic pump for the controlled release of a pharmaceutically active agent, consisting essentially of a core containing an active agent and a rate-controlling water-insoluble wall comprising a semi-permeable polymer and at least one pH insensitive pore-forming additive dispersed throughout the wall.

U.S. Pat. No. 4,590,062 discloses a compressed product containing an active agent produced by dry blending with a matrix combination of a hydrophobic polymer such as ethylcellulose, and a wax, fatty acid, neutral lipid or combination thereof.

U.S. Pat. No. 4,996,047 is directed to an oral pharmaceutical composition in unit dosage form of ion-exchange resin particles having a pharmacologically active drug bound thereto, wherein the drug-resin complex particles are coated with a water-impermeable diffusion barrier to provide controlled release of the active drug.

U.S. Pat. No. 5,120,548 discloses a controlled-release drug delivery device comprising a polymer composition which swells upon exposure to an aqueous environment, a plurality of controlled-release swelling modulators, at least one active agent and a water insoluble polymer or a microporous wall surrounding the composition.

U.S. Pat. No. 5,350,584 discloses a process for the production of microcrystalline cellulose-free multiparticulates comprising a medicament and a charged resin, producing spheronized beads that can be used in controlled-release dosage forms.

U.S. Pat. No. 5,366,738 describes a drug delivery device for controlled release of an active agent, including a compressed core with an active agent and a polymer that forms gelatinous microscopic particles upon hydration and a water insoluble, water impermeable polymeric coating comprising a polymer and plasticizer that surrounds and adheres to the core.

U.S. Pat. No. 5,582,838 discloses a drug delivery device for the controlled release of a beneficial agent, including a compressed core having at least two layers. At least one layer is a mixture of a beneficial agent and a polymer that forms microscopic polymer gel beads upon hydration and at least one outer layer comprises a polymer that forms microscopic polymer gel beads upon hydration. A water-insoluble, water-impermeable coating is applied to the core. The coating has apertures exposing between about 5-75% of the core surface.

U.S. Pat. No. 5,874,418 discloses a pharmaceutical composition comprising a carrier and a mixture of a sulfoalkyl ether-cyclodextrin and a therapeutic agent. Delayed, sustained or controlled release formulations are described wherein the pharmaceutical core is coated with a film coating comprising a film forming agent and a pore forming agent.

U.S. Pat. No. 5,882,682 relates to a drug delivery process involving: preparing a uniform mixture of a polymer that forms gelatinous microscopic particles upon hydration, the beneficial agent and other excipients; compressing the mixture into cores; coating the cores with a water-insoluble, water-impermeable polymeric coating including a polymer and a plasticizer; and forming apertures through the coating.

U.S. Pat. No. 5,952,451 relates to a process for preparing high molecular weight poly(phosphoester) compositions comprising a biologically active substance, useful in prolonged released drug delivery systems.

A multi-layered osmotic device described in U.S. Pat. No. 6,004,582 comprises a compressed core including a first active agent and an osmotic agent. A semi-permeable membrane having a preformed passageway therein surrounds the core, wherein the membrane is permeable to a fluid and is substantially impermeable to the first active agent. This membrane preferably consists essentially of cellulose acetate and poly(ethylene glycol). The external coat can include poly(vinylpyrrolidone) and poly(ethylene glycol) and further materials such as HPMC, ethylcellulose, hydroxylethylcellulose, CMC, dimethylaminoethyl methacrylate-methacrylic acid ester copolymer, ethyl acrylate-methyl methacrylate copolymer, and combinations thereof.

As mentioned above, U.S. Pat. No. 7,387,793 proposes an extended release form of cyclobenzaprine for treating muscle spasm associated with painful musculoskeletal conditions, wherein the extended-release is produced by beads comprising the cyclobenzaprine coated with a water-insoluble polymer possibly in combination with a water-soluble polymer.

WO 99/30671 describes an oral delivery vehicle including an aspected particle comprising a pharmaceutically active component and excipients and a coating to provide sustained drug delivery to the particle.

WO 98/53802 describes a multi-layered osmotic device capable of delivering a first active agent in an outer lamina to one use environment and a second active agent in the core to another use environment. An erodible polymer coat between an internal semi permeable membrane and a second active agent-containing external coat comprises poly(vinylpyrrolidone)-vinyl acetate) copolymer. The active agent in the core is delivered through a pore containing an erodible plug.

WO 98/18610 relates to particles containing an active agent, which provide controlled release of the active ingredient without substantial destruction of the matrix material. A release-rate controlling component is incorporated in a matrix to control the rate-release of the encapsulant. A hydrophobic component or a high water binding capacity component may be used for extending the release time. Release properties may also be controlled by precoating the encapsulant and/or coating the particles with a film-forming component.

WO 98/06439 relates to a composition comprising a biologically active agent encapsulated in a matrix comprising a polyether ester copolymer, such as polyethylene glycol terephthalate/polybutylene-terephthalate copolymer, that protects the active agent from degradation and facilitates the drug delivery.

### SUMMARY OF THE INVENTION

One aspect of the invention provides an extended-release dosage form of cyclobenzaprine for use in the treatment of tinnitus and related auditory dysfunctions by once-a-day oral administration, wherein the dosage form is a tablet or capsule comprising cyclobenzaprine as active agent in an amount from 10-80 mg, preferably from 10-60 mg, 10-50 mg, or 15-45 mg, the active agent being associated with a polymer coating or matrix that comprises a water-insoluble polymer, the polymer coating or matrix providing the dosage form with an extended release of the active agent over at least 12 hours and preferably over at least 16 hours when the dosage form is administered to a patient.

As explained below, this combines the efficacy of cyclobenzaprine for treating tinnitus and related auditory dysfunctions with an improvement in the desired relatively uniform plasma concentrations leading to maximum efficacy while reducing side effects and minimizing intersubject variability.

Providing the dosage form with an extended release of the active agent over at least 12 hours when the dosage form is administered to a patient means that usually from 60-85% of the total active agent is released gradually over 12 hours, under standard conditions. In particular, in most cases the polymer coating or matrix provides the dosage form with the following dissolution profile when measured in a USP type II apparatus at 50 rpm, at a temperature of 37 degrees C.:
no more than about 40% of the total active agent is released in 2 hours;
from about 40-65% of the total active agent is released after 4 hours;
from about 60-85% of the total active agent is released after 8 hours; and
from about 75-85% of the total active agent is released after 12 hours.

After 16 hours, typically more than 85% of the total active agent will be released.

The water insoluble polymer is usually selected from ethers of cellulose, esters of cellulose, cellulose acetate, ethyl cellulose, polyvinyl acetate, neutral copolymers based on ethylacrylate and methylmethacrylate, copolymers of acrylic and methacrylic acid esters with quaternary ammonium groups, pH-insensitive ammonio methacrylic acid copolymers, and mixtures thereof.

The polymer coating or matrix may further comprise a water-insoluble polymer for example selected from methylcellulose, hydroxypropylcellulose, hydroxypropyl methylcellulose, polyethylene glycol polyvinylpyrrolidone and mixtures thereof.

The polymer coating or matrix can further comprise a plasticizer, in particular selected from triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

The extended-release dosage forms described herein can comprise a polymer coating on core particles comprising the active agent, wherein the water-insoluble polymer in the polymer coating corresponds for example to 7% to 12% of the weight of the dosage form. The core particles may be formed by the active agent alone or coated on an inert particle core in particular a sugar sphere or an acidic or alkaline buffer crystal.

The particles may be formed by granulating and dry milling and/or by extrusion and spheronization of the active agent, then applying a polymer coating.

The dosage form can alternatively be formed into capsules containing extended-release coated particles as described above, or a mixture of extended-release coated particles and “immediate release” particles of the uncoated active agent, and/or a mixture of extended-release coated particles having different coatings providing different release times.

Instead of including coated particles, the dosage form may comprise the active agent cyclobenzaprine associated with a polymer matrix as described in several of the prior art disclosures summarized above.

The active agent can be included in the dosage form as cyclobenzaprine or as pharmaceutically acceptable salts and derivatives thereof, in particular as cyclobenzaprine hydrochloride.

The extended-release dosage form can be used for a long-term treatment of tinnitus and related auditory dysfunctions extending over 2 weeks or more, preferably 8 weeks or more.

Another aspect of the invention is a method of treating tinnitus and related auditory disorders in a mammal, comprising once-a-day orally administering to a mammal in need of such treatment, an extended-release dosage form of cyclobenzaprine, wherein the dosage form is a tablet or capsule comprising cyclobenzaprine as active agent in an amount from 10-80 mg, preferably from 10-60 mg, the active agent being associated with a polymer coating or matrix that comprises a water-insoluble polymer, the polymer coating or matrix providing the dosage form with an extended release of the active agent over at least 12 hours and preferably over at least 16 hours when the dosage form is administered to a patient. This method is in particular applicable to a subject having been previously diagnosed with and possibly also treated for tinnitus.

As shown by the tests reported below, which are taken over from the above-mentioned PCT patent application PCT/IB2010/051373, cyclobenzaprine has a positive effect on tinnitus severity and on tinnitus loudness in the tested subjects, it is safe to administer and though common side effects (like constipation and dry mouth) may be experienced, it is tolerated well by most subjects. Similar results are expected for associated auditory dysfunctions. Generally, according to the invention, the described extended release form of cyclobenzaprine is effective for the treatment of an auditory dysfunction selected from tinnitus, hyperacusis, auditory hallucinations, misophonia, phonophobia and central auditory processing disorders.

General Aspects of Extended-Release Cyclobenzaprine for Treating Tinnitus

For many chronic conditions such as chronic pain, management guidelines recommend the use of long-acting, extended-release formulations. Guidelines for pharmacological treatment of tinnitus however have not been established, although tinnitus is a chronic condition. As such, the goal of pharmacological therapy for tinnitus is to provide sustained relief. The use of long-acting, extended-release formulations for tinnitus is desirable because they provide prolonged, more consistent plasma concentrations of drug compared with short-acting agents, thus minimizing fluctuations that could contribute to end-of-dose breakthrough tinnitus. In this regard, a randomized, open-label, two-period crossover, single-centre study, has demonstrated that single-dose pharmacokinetics of once-daily cyclobenzaprine extended release 30 mg versus cyclobenzaprine immediate release 10 mg three times daily in healthy young adults, provides a controlled release of cyclobenzaprine with sustained plasma concentrations, in contrast to the fluctuating profile of cyclobenzaprine immediate release with comparable systemic exposures.

Better Efficacy and Fewer Side Effects

With cyclobenzaprine extended release a more stable plasma concentration can be achieved. In general, in chronic diseases, less fluctuations in the plasma concentrations result in better efficacy. This is well known for the treatment of chronic pain, where analgesics are most efficient when they are administered in a way which results in a very stable plasma concentration, e.g. by transdermal application.

For example, side effects of tricyclic drugs depend more on changes in the plasma concentration and on peak concentration than on the mean concentration over time. So every drug administration regime which results in more stable plasma concentrations and fewer fluctuations will produce less side effects. In the case of cyclobenzaprine, constipation, which is the main complaint and the main reason for dropouts from therapy, could be diminished.

Control of Tinnitus Overnight

The patient with tinnitus is burdened by decreased quality of life, decreased sleep, interference with social relationships, diminished cognitive functions, interference with activities of daily living, decreased productivity, and increased anxiety and depression. Tinnitus can disrupt sleep, and poor sleep can lower the tinnitus threshold, which may contribute to increased tinnitus. Thus, effective management of tinnitus is complicated by the presence of additional conditions that occur frequently together with tinnitus, in particular sleep disturbances. The most frequent sleep complaints include delayed onset of sleep, frequent awakenings, decreased sleep duration, daytime fatigue, and non-restorative sleep. Because greater tinnitus annoyance has been associated with decreased sleep satisfaction, less total sleep time, delayed onset of sleep, and more awakenings due to tinnitus, effective tinnitus control should improve sleep in patients with tinnitus and vice versa. Thus, an important goal of tinnitus pharmacotherapy is to provide sustained relief; therefore, regular administration of immediate release formulations is required to ensure that the next dose of medication is given before the effects of the previous dose have dissipated. Extended release formulations in general provide more consistent and improved nighttime control, without the need to take another dose of medication during the night, and less clock-watching by patients. All these aspects are expected to improve tinnitus-related sleep disturbances.

Compliance

In order to achieve sufficient stable plasma concentrations with short-acting (immediate release) cyclobenzaprine, the drug has to be taken at least three times daily. In contrast, a once daily regimen suffices for the extended release formulation of cyclobenzaprine. Consequently, tinnitus patients would benefit enormously from an extended-release formulation, which can be administered once daily. Among the factors which are most important for long term efficiency is treatment adherence and compliance. For long-term compliance of drug intake the administration regime is of utmost importance. The patient compliance for a once-daily administration is much higher than for a treatment regimen which requires drug intake three times a day.

### SUMMARY OF ADVANTAGES

Extended release formulations provide better around-the-clock efficacy, result in fewer changes in drug plasma concentrations when compared with short-acting formulations, provide maximal tolerability, and have minimal long term adverse events with prolonged use.

The total dosage needed for achieving symptom control is lower, the side effects which depend on peak concentrations and on concentration changes are fewer and less pronounced. At equal concentrations, extended release cyclobenzaprine is more efficient.

In addition, extended release formulations should provide better nighttime tinnitus control with less need for nighttime dosing, improving sleep. Finally, compliance and long-term adherence are better, resulting in better long-term efficiency.

### BRIEF DESCRIPTION OF THE DRAWINGS

FIG. 1 is a graph of the Global Improvement Scale of the tested subjects.

FIG. 2-6 are graphs showing the audiogram of different subjects; the progression of Minimum Masking Levels (MML) during the tests; and the progression of Tinnitus Handicap Inventory (THI) and Tinnitus Impairment Questionnaire (TBF12) during the tests.

### DETAILED DESCRIPTION

As described in U.S. Pat. No. 7,387,793, the contents whereof are incorporated herein by way of reference, the dosage form of the present invention may include particles with an active core that comprises an inert particle or an acidic or alkaline buffer crystal, which is coated with a active-agent-containing film-forming formulation, preferably a water-soluble film-forming composition to form a water-soluble/dispersible particle.

The amount of active agent in the core will depend on the required dose, and usually varies from about 5 to 60 weight %. Generally, the polymeric coating on the active core will be from about 4 to 20% based on the weight of the coated particle, depending on the type of release profile required and/or the polymers and coating solvents chosen. In one embodiment, the inactive core may be a sugar sphere or a buffer crystal or an encapsulated buffer crystal such as calcium carbonate, sodium bicarbonate, fumaric acid, tartaric acid which alters the microenvironment of the active agent to facilitate its release.

Instead of coating the active agent on an inert core, alternatively the active agent may be prepared by granulating and milling and/or by extrusion and spheronization of a polymer composition containing the active substance.

The active-agent-containing particle may be coated with an extended release coating comprising a water insoluble polymer or a combination of a water insoluble polymer and a water soluble polymer to provide extended release beads. In certain embodiments, the water insoluble polymer and the water soluble polymer may be present at a weight ratio of from 100/0 to 65/35, more particularly from about 95/5 to 70/30, and still more particularly at a ratio of from about 85/15 to 75/25. The extended release coating is applied in an amount necessary to provide the desired release profile and typically comprises from about 1% to 15%, more particularly from about 7% to 12%, by weight of the coated beads.

The invention also contemplates a modified release dosage form including a mixture of two bead populations. One method of manufacturing such a mixture includes the steps of: 1. preparing a drug-containing core by coating an inert particle such as a non-pareil seed, an acidic buffer crystal or an alkaline buffer crystal with the active agent and a polymeric binder or by granulation and milling or by extrusion/spheronization to form an immediate-release bead; 2. coating the immediate-release bead with a plasticized water-insoluble polymer alone such as ethylcellulose or in combination with a water soluble polymer such as hydroxypropylmethylcellulose to form an extended release bead; 3. filling into hard gelatin capsules extended-release beads alone or in combination with immediate-release beads at a ratio to produce modified release capsules providing the desired release profile.

An aqueous or a pharmaceutically acceptable solvent medium may be used for preparing active-agent-containing core particles. The type of film-forming binder that is used to bind the drug to the inert sugar sphere is not critical but usually water soluble, alcohol soluble or acetone/water soluble binders are used. Binders such as polyvinylpyrrolidone (PVP), polyethylene oxide, hydroxypropyl methylcellulose (HPMC), hydroxypropylcellulose (HPC), polysaccharides such as dextran, corn starch may be used at concentrations for example from about 0.5 to 5 weight %. The active agent may be present in this coating formulation in the solution form or may be dispersed at a solid content up to about 35 weight % depending on the viscosity of the coating formulation.

In certain embodiments, the active substance, optionally a binder such as PVP, a dissolution rate controlling polymer (if used), and optionally other pharmaceutically acceptable excipients are blended together in a planetary mixer or a high shear granulator such as from Aeromatic-Fielder (GSA Pharma Systems AG) and granulated by adding/spraying a granulating fluid such as water or alcohol. The wet mass can be extruded and spheronized to produce spherical particles (beads) using an extruder/marumerizer. In these embodiments, the drug load could be as high as 90% by weight based on the total weight of the extruded/spheronized core.

Cyclobenzaprine hydrochloride is normally used as active agent.

Representative examples of water insoluble polymers useful in the extended-release coating include ethylcellulose powder or an aqueous dispersion (such as AQUACOAT™. ECD-30), cellulose acetate, polyvinyl acetate (Kollicoat SR#30D from BASF), neutral copolymers based on ethyl acrylate and methylmethacrylate, copolymers of acrylic and methacrylic acid esters with quaternary ammonium groups such as Eudragit NE, RS and RS30D, RL or RL30D and the like. Representative examples of water soluble polymers include low molecular weight hydroxypropyl methylcellulose (HPMC), methylcellulose, hydroxypropylcellulose, polyvinylpyrrolidone, polyethylene glycol (PEG of molecular weight>3000) and mixtures thereof. The extended release coating will typically be applied at a thickness ranging from about 1 weight % up to 15 weight % depending on the solubility of the active in water and the solvent or latex suspension-based coating formulation used.

The coating compositions used in forming the membranes are usually plasticized. Representative examples of plasticizers include triacetin, tributyl citrate, triethyl citrate, acetyl tri-n-butyl citrate diethyl phthalate, polyethylene glycol, polypropylene glycol, castor oil, dibutyl sebacate, acetylated monoglycerides and the like or mixtures thereof. The plasticizer may comprise about 3 to 30 wt. % and typically about 10 to 25 wt. % based on the polymer. The type of plasticizer and its content depends on the polymer or polymers, and the nature of the coating system (e.g., aqueous or solvent-based, solution or dispersion-based and the total solids).

In general, it is desirable to prime the surface of the particle before applying an extended release membrane coating or to separate the different membrane layers by applying a thin hydroxypropyl methylcellulose (HPMC)(OPADRY™ Clear) film. While HPMC is typically used, other primers such as hydroxypropylcellulose (HPC) can also be used.

The membrane coatings can be applied to the core using any of the coating techniques commonly used in the pharmaceutical industry, particularly fluid bed coating.

The present invention is applied to multi-dose forms, i.e., active agent products in the form of multi-particulate dosage forms (pellets, beads, granules or mini-tablets) or in other forms suitable for oral administration. As used herein, these terms refer interchangeably to multi-particulate dosage forms.

The invention will further be described by way of example in the tests reported below for tinnitus.

### DESCRIPTION AND RESULTS OF TRIAL TESTS

From February to November 2008, 30 subjects were screened and 15 recruited to this trial. The sample counted on 7 males and 8 females with an age range from 36 to 71 years, mean age 54.6 years (Std=11.3 years).

Medication

The selected subjects received Cyclobenzaprine tablets, in the amounts described below. In the initial tests as reported in PCT/IB2010/051373 tablets were administered three times daily. However short-term tests using 15 mg and 30 mg extended-release cyclobenzaprine available under the trademark Amrix give equally encouraging results in terms of tinnitus treatment. From theoretical considerations and from the data provided in U.S. Pat. No. 7,387,793, extended-release cyclobenzaprine is expected to provide the same tinnitus-treating effect combined with an improvement in the desired relatively uniform plasma concentrations leading to maximum efficacy while reducing side effects and minimizing intersubject variability.

Table 1a shows the cyclobenzaprine dosage 3 times/day and Table 1b shows the proposed cyclobenzaprine dosage with extended-release cyclobenzaprine 1 time/day.

TABLE 1a
Cyclobenzaprine dosage

| | Baseline- | Week | Week | Week | Week | Week | Week |
|---|---|---|---|---|---|---|---|
| | week 2 | 2-4 | 4-8 | 8-12 | 12-13 | 13-14 | 14-16 |
| Concentration | 15 | 22.5 | 30 | 30 | 22 5 | 15 | 0 |
| mg/day | | | | | | | |

TABLE 1b
Extended-release cyclobenzaprine proposed dosage

| | Baseline- | Week | Week | Week | Week |
|---|---|---|---|---|---|
| | week 3 | 4-8 | 8-12 | 13-14 | 14-16 |
| Concentration | 15 | 30 | 30 | 15 | 0 |
| mg/day | | | | | |

Results

Four selected outcome variables were used in this preliminary analysis: Global improvement (GI), Tinnitus Handicap Inventory (THI) and Tinnitus Impairment Questionnaire (TBF 12) questionnaires and Minimum Masking Levels (MML). The changes on the outcome variables were obtained by subtracting week 12's scores from the screening scores. The presence of a negative result means a decrease on values. On Subject number 6 the final scores correspond to week 8 (last observation carried forward analysis). Subject 3 had an intermittent tinnitus which interfered on the psychoacoustic examination.

TABLE 2
Changes on outcome variables ordered by
Global Improvement (GI) classification.

| | Changes on | | | | |
|---|---|---|---|---|---|
| Subject | | | | MML | |
| number | GI | THI | TBF 12 | Right | Left |
| 2 | 1 | −14 | −2 | −5 | −8 |
| 3 | 1 | −30 | 6 | Not possible to measure but noticed | |
| | | | | change on intensity and less episodes | |
| | | | | during the day | |
| 8 | 2 | −48 | −11 | −11 | −17 |
| 4 | 2 | −8 | −3 | −25 | −12 |
| 10 | 2 | −14 | −2 | −8 | −9 |
| 14 | 2 | −10 | −10 | 2 | 0 |
| 11 | 2 | −28 | −5 | −5 | 1 |
| 13 | 2 | −40 | −7 | 1 | −8 |
| 9 | 3 | −14 | −1 | −7 | −7 |
| 5 | 3 | −42 | −4 | 0 | −4 |
| 6 | 3 | −38 | −4 | Trial suspended because of side effects | |
| 15 | 4 | −36 | −6 | Didn't return for measurements | |
| 7 | 4 | −32 | −3 | 9 | −7 |
| 12 | 4 | −28 | −6 | 3 | 8 |
| 1 | 4 | 0 | −3 | 14 | 3 |

In Table 2 the changes on outcome variables are listed by Global Improvement (GI) classification, as follows: GI 1=much better; 2=better; 3=slightly better; 4=no change; 5=slightly worse; 6=worse; 7=much worse; THI: Tinnitus Handicap Inventory, TBF 12: Tinnitus Impairment Questionnaire; MML: minimal masking levels, measured in dB HL (Hearing Level).

Four different criteria were established to determine whether a subject responded or not to treatment (see Table 3). The prevalence of positive results to Cyclobenzaprine will reflect on how rigid the outcome criteria are selected.

TABLE 3
Criteria and prevalence of Cyclobenzaprine treatment results.

| | | Non | |
|---|---|---|---|
| | Responders | responders | Responders |
| Criteria | % and n | % and n | (subject #) |
| Criteria 1 | 73.3% | 26.7% | 2, 3, 4, 5, 6, 8, 9, |
| Improvement on GI | 11 | 4 | 10, 11, 13 and 14 |
| Criteria 2 | 73.3% | 26.4% | 2, 3, 4, 5, 6, 8, 9, |
| Criteria 1 + decrease on | 11 | 4 | 10, 11, 13 and 14 |
| THI | | | |
| Criteria 3 | 66.6% | 33.3% | 2, 4, 5, 6, 8, 9, |
| Criteria 2 + decrease on | 10 | 5 | 10, 11, 13 and 14 |
| TBF 12 | | | |
| Criteria 4 | 53.3% | 46.7% | 2, 4, 5, 8, 9, 10, |
| Criteria 3 + decrease on | 8 | 7 | 11 and 13 |
| MML | | | |

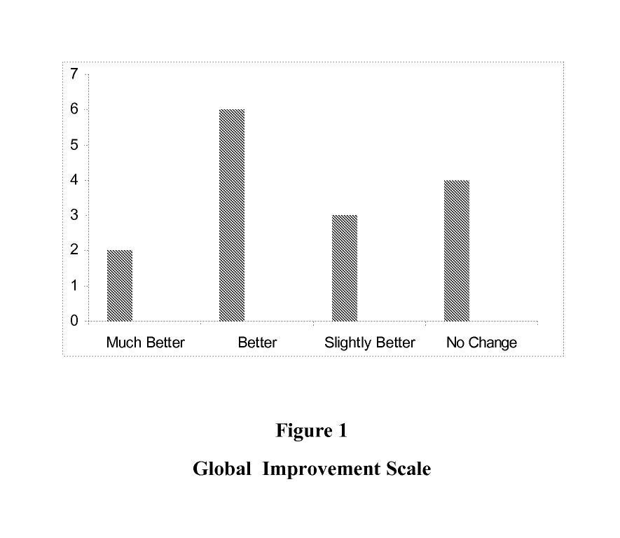

Using the less conservative criteria, Criteria 1, based on subjects score to Global Improvement, (GI) 11 subjects (73.3%) referred that tinnitus improved with medication and 4 (26.7%) didn't notice any change. Different degrees of changes in GI perception are described on FIG. 1. As can be seen, 8 subjects were Better or Much Better; and 11 subjects were Slightly Better, Better or Much Better.

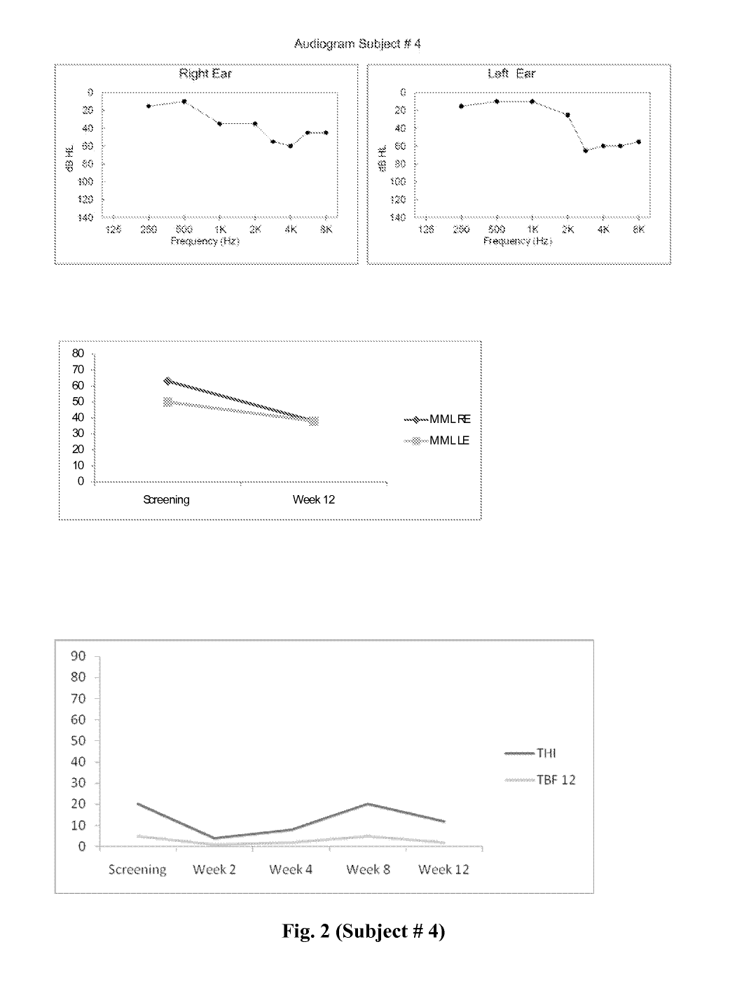

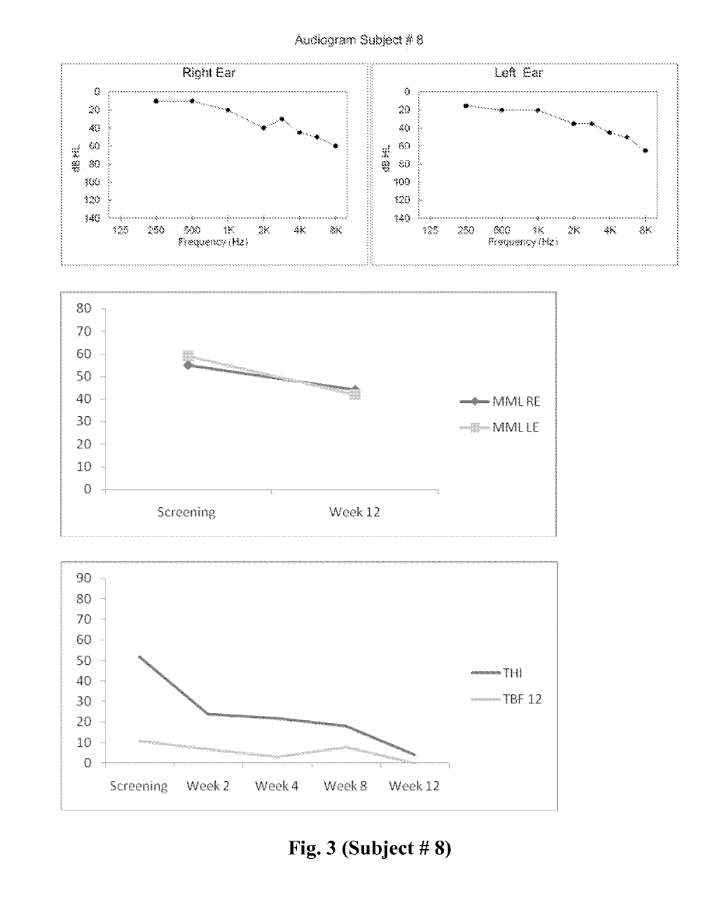

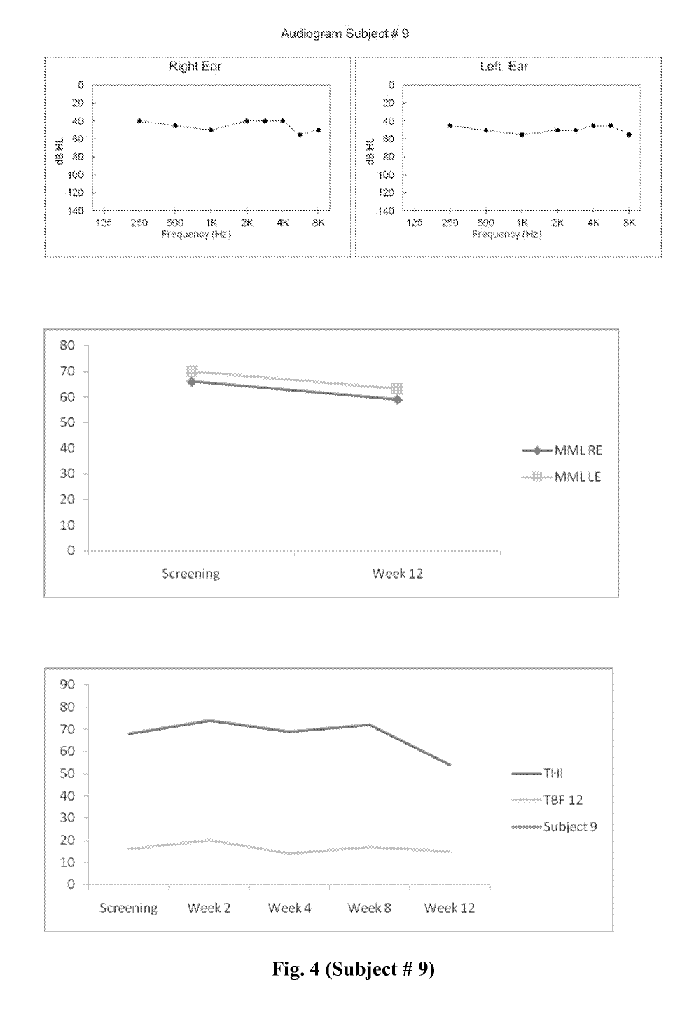

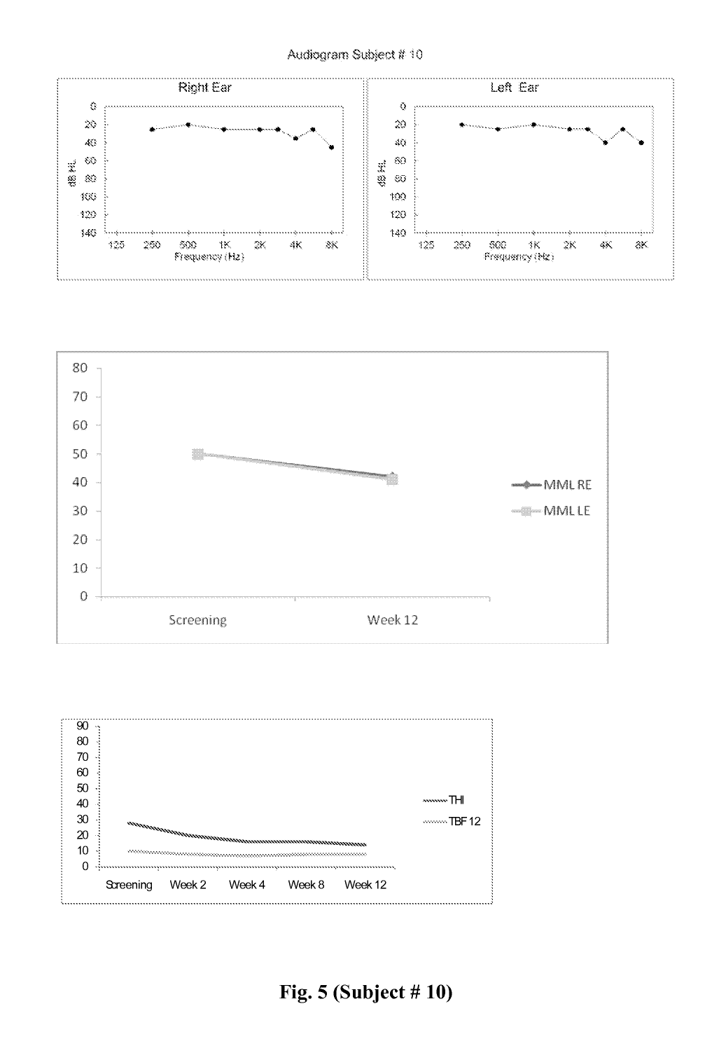

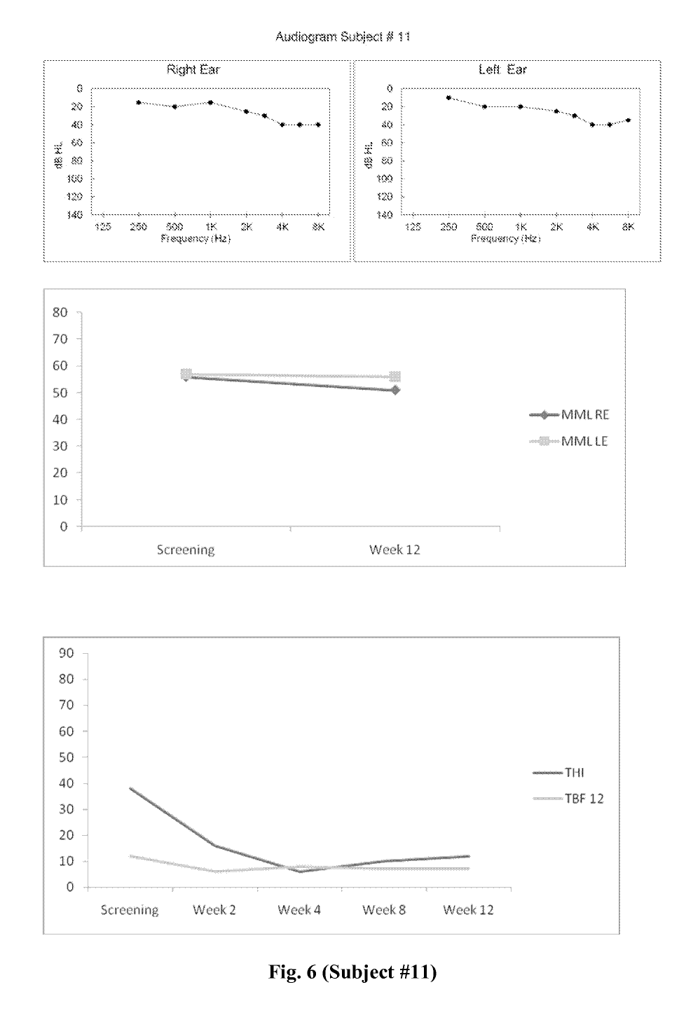

FIGS. 2-6 show by way of example results for subjects 4, 8, 9, 10 and 11 that responded to all of the Criteria 1-4. The progressive reduction of the Minimum Masking Levels (MML) in all of these subjects is particularly striking. Using Criteria 4, the most conservative approach to select responders, only subjects that reported improvement on GI and demonstrated decrease on THI, TBF 12 and MML were included (see Table 2). Based on these criteria the rate of improvement was 53.3%.

General Data on Subjects and Tinnitus Characteristics

Some of the data collected on this trial are listed on Tables 4 to 7, so one can have an idea about the bibliographical data, tinnitus characteristics and other variables collected on Case History Questionnaire. Tinnitus psychoacoustic measurements, audiograms as well as time line graphs for selected subjects showing THI and TB12 during different moments at the trial are presented in FIGS. 2 to 6.

TABLE 4
Case History Questionnaire Data of the 15 subjects

| | | | | | | Time of | Gradual/ | |
|---|---|---|---|---|---|---|---|---|
| Subject | | | Family | | | onset | sudden | |
| number | Gender | Age | history | Etiology | Hearing | (months) | onset | Pulse |
| 1 | M | 62 | Y | Other | Asymmetric | 180 | Gradual | N |
| | | | | | NSHL* | | | |
| 2 | M | 43 | N | Other | Unilateral | 10 | Gradual | N |
| | | | | | NSHL Left | | | |
| 3 | F | 57 | Y | Other | Unilateral | 180 | Sudden | YES, ≠ |
| | | | | | NSHL Right | | | heart |
| 4 | M | 70 | Y | Noise | Asymmetric | 72 | Gradual | N |
| | | | | | NSHL | | | |
| 5 | F | 61 | N | Stress | Asymmetric | 60 | Gradual | Y, Like |
| | | | | | NSHL | | | heart |
| 6 | F | 36 | Y | Ear | Normal | 8 | Gradual | Y, Like |
| | | | | Infection | | | | heart |
| 7 | M | 60 | N | Noise | Symmetric | 504 | Gradual | N |
| | | | | | NSHL | | | |
| 8 | F | 71 | N | Other | Symmetric | 336 | Gradual | N |
| | | | | | NSHL | | | |
| 9 | F | 57 | Y | Other | Asymmetric | 336 | Gradual | N |
| | | | | | NSHL | | | |
| 10 | F | 69 | N | Other | Symmetric | 216 | Gradual | N |
| | | | | | NSHL | | | |
| 11 | F | 43 | N | Hearing | Symmetric | 24 | Sudden | N |
| | | | | Loss | NSHL | | | |
| 12 | F | 56 | N | Hearing | Asymmetric | 144 | Gradual | N |
| | | | | loss | NSHL | | | |
| 13 | M | 40 | N | Noise | Symmetric | 216 | Gradual | Y |
| | | | | | NSHL | | | |
| 14 | M | 45 | N | Noise | Asymmetric | 288 | Gradual | N |
| | | | | | NSHL | | | |
| 15 | M | 49 | N | Barotrauma | Unilateral | 8 | Gradual | N |
| | | | | | NSHL Left | | | |

*NSHL = neurosensory hearing loss

TABLE 5
Tinnitus description from Case History Questionnaire

| Subject | | Constant/ | Changes On | Intensity | | |
|---|---|---|---|---|---|---|
| number | Location | intermittent | intensity | 0-100 | Quality | Pitch |
| 1 | BOTH EARS | Constant | Y | 30 | Crickets | High |
| 2 | LEFT EAR | Intermittent | Y | 50 | Wheezing | High |
| 3 | RIGHT EAR | Intermittent | Y | 70 | Crickets | Medium |
| 4 | BOTH EARS | Constant | Y | 30 | Crickets | High |
| 5 | BOTH EARS, | Constant | Y | 70 | Wheezing | Very High |
| | WORST LEFT | | | | | |
| 6 | BOTH EARS, | Constant | Y | 50 | Wheezing/ | High |
| | WORST LEFT | | | | crickets | |
| 7 | BOTH EARS | Constant | Y | 70 | Wheezing | Medium |
| 8 | LEFT EAR | Constant | N | 60 | Wheezing | Medium |
| 9 | BOTH EARS | Constant | Y | 80 | Tonal | High |
| 10 | BOTH, WORST | Constant | N | 90 | Wheezing | High |
| | RIGHT | | | | | |
| 11 | BOTH, | Constant | N | 80 | Other | High |
| | WORST RIGHT | | | | | |
| 12 | LEFT EAR | Intermittent | Y | 70 | Wheezing | Very High |
| 13 | BOTH EARS, | Constant | N | 50 | Wheezing | Medium |
| | WORST LEFT | | | | | |
| 14 | BOTH, | Constant | N | 60 | Other | Very High |
| | WORST RIGHT | | | | | |
| 15 | LEFT EAR | Constant | N | 70 | Crickets | High |

TABLE 6
Associated Symptoms/Somatic modulation

| Subject | | | TMJ* | Neck | Other | Somatic |
|---|---|---|---|---|---|---|
| # | Headache | Vertigo | Dysfunction | Pain | Pain | modulation |
| 1 | N | N | N | Y | Y | N |
| 2 | N | N | N | Y | Y | N |
| 3 | N | N | N | Y | N | N |
| 4 | Y | Y | N | Y | Y | Y |
| 5 | N | N | N | Y | Y | N |
| 6 | Y | Y | Y | Y | Y | Y |
| 7 | Y | Y | Y | Y | Y | N |
| 8 | N | N | Y | Y | Y | N |
| 9 | Y | Y | Y | N | Y | N |
| 10 | N | N | N | N | Y | N |
| 11 | Y | Y | Y | Y | Y | N |
| 12 | Y | N | N | N | N | Y |
| 13 | Y | Y | N | Y | N | N |
| 14 | N | N | Y | Y | N | Y |
| 15 | N | N | Y | Y | Y | Y |

*Temporomandibular Joint Dysfunction

TABLE 7
Tinnitus Psychoacoustic characteristics collected at screening and at week 12
(“12 W”)

| | | | MML Right Ear | | MML Left Ear | | Residual | |
|---|---|---|---|---|---|---|---|---|
| | Pitch (kHz) | | (dB HL) | | (dB HL) | | Inhibition | |
| Subject # | Screening | 12 W | Screening | 12 W | Screening | 12 W | Screening | 12 W |
| 1 | 2 | 3 | 38 | 52 | 42 | 45 | Complete | No |
| 2 | Broad Band | Broad Band | 50 | 45 | 70 | 62 | Complete | Complete |
| 3 | — | 6 | — | — | — | — | — | — |
| 4 | 8 | 4 | 63 | 38 | 50 | 38 | Complete | Partial |
| 5 | 4 | 4 | 53 | 53 | 49 | 45 | Complete | Complete |
| 6 | Broad Band | — | 15 | . | 24 | . | No | — |
| 7 | 2 | 2 | 35 | 44 | 44 | 37 | Partial | Partial |
| 8 | 6 | Broadband | 55 | 44 | 59 | 42 | Partial | Complete |
| 9 | 4 | 6 AND 1 | 66 | 59 | 70 | 63 | Complete | Complete |
| 10 | 8 | Broadband | 50 | 42 | 50 | 41 | Complete | Complete |
| 11 | 8 | 4 | 56 | 51 | 57 | 56 | Partial | Partial |
| 12 | Broadband | Broadband | 40 | 43 | 37 | 45 | Complete | Complete |
| | +2K | +2K | | | | | | |
| 13 | 8 | 8 | 15 | 16 | 25 | 17 | Complete | Complete |
| 14 | 4 | 4 | 35 | 37 | 34 | 34 | Partial | Partial |
| 15 | 6 | | 40 | . | 57 | . | Complete | — |

Changes on tinnitus pitch were an unexpected effect of the tests, probably indicating that this medication induced a change on neural firing pattern. It was observed in 5 out of 15 subjects.

The time of tinnitus onset was not a predictive variable to positive outcome; subjects suffering for 28 years had positive results as well as subjects who had it for 8 months.

All publications, patent applications, patents, and other documents cited herein are incorporated by reference in their entirety. In case of conflict, the present specification, including definitions, will control. In addition, the materials, methods, and examples are illustrative only and not intended to be limiting in any way.

1. An extended-release dosage form of cyclobenzaprine for use in the treatment of tinnitus and tinnitus-related auditory dysfunctions including hyperacusis, auditory hallucinations, misophonia, phonophobia and central auditory processing disorders, but excluding the condition known as “exploding head syndrome” also referred to as “explosive tinnitus”, by once-a-day oral administration, wherein the dosage form is a tablet or capsule comprising cyclobenzaprine as active agent in an amount from 10-80 mg, preferably from 10-60 mg, the active agent being associated with a polymer coating or matrix that comprises a water-insoluble polymer, the polymer coating or matrix providing the dosage form with an extended release of the active agent over at least 12 hours and preferably over at least 16 hours when the dosage form is administered to a patient.

2. The extended-release dosage form for use in the treatment of tinnitus and tinnitus-related auditory dysfunctions as claimed in claim 1, wherein the active agent cyclobenzaprine is present in an amount of 10-50 mg, preferably 15-45 mg.

3. The extended-release dosage form for use in the treatment of tinnitus and tinnitus-related auditory dysfunctions as claimed in claim 1, wherein the polymer coating or matrix provides the dosage form with the following dissolution profile when measured in a USP type II apparatus at 50 rpm, at a temperature of 37 degrees C.:
no more than about 40% of the total active agent is released in 2 hours;
from about 40-65% of the total active agent is released after 4 hours;
from about 60-85% of the total active agent is released after 8 hours; and
from about 75-85% of the total active agent is released after 12 hours.

4. The extended-release dosage form for use in the treatment of tinnitus and tinnitus-related auditory dysfunctions as claimed in claim 1, wherein said water insoluble polymer is selected from ethers of cellulose, esters of cellulose, cellulose acetate, ethyl cellulose, polyvinyl acetate, neutral copolymers based on ethylacrylate and methylmethacrylate, copolymers of acrylic and methacrylic acid esters with quaternary ammonium groups, pH-insensitive ammonio methacrylic acid copolymers, and mixtures thereof.

5. The extended-release dosage form for use in the treatment of tinnitus and tinnitus-related auditory dysfunctions as claimed in claim 1, wherein the polymer coating or matrix further comprises a water-insoluble polymer.

6. The extended-release dosage form for use in the treatment of tinnitus and tinnitus-related auditory dysfunctions as claimed in claim 5, wherein the water soluble polymer is selected from methylcellulose, hydroxypropylcellulose, hydroxypropyl methylcellulose, polyethylene glycol polyvinylpyrrolidone and mixtures thereof.

7. The extended-release dosage form for use in the treatment of tinnitus and tinnitus-related auditory dysfunctions as claimed in claim 1, wherein the polymer coating or matrix further comprises a plasticizer, in particular selected from triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

8. The extended-release dosage form for use in the treatment of tinnitus and tinnitus-related auditory dysfunctions as claimed in claim 1, comprising a polymer coating on core particles that comprise the active agent.

9. The extended-release dosage form for use in the treatment of tinnitus and tinnitus-related auditory dysfunctions as claimed in claim 1, wherein the core particles are formed by the active agent alone or coated on an inert particle core.

10. The extended-release dosage form for use in the treatment of tinnitus and tinnitus-related auditory dysfunctions as claimed in claim 1 for a long-term treatment of tinnitus and said related auditory dysfunctions extending over 2 weeks or more, preferably 8 weeks or more.

11. A method of treating tinnitus and tinnitus-related auditory dysfunctions including hyperacusis, auditory hallucinations, misophonia, phonophobia and central auditory processing disorders, but excluding the condition known as “exploding head syndrome” also referred to as “explosive tinnitus”, in a mammal, comprising once-a-day orally administering to a mammal in need of such treatment, an extended-release dosage form of cyclobenzaprine, wherein the dosage form is a tablet or capsule comprising cyclobenzaprine as active agent in an amount from 10-80 mg, preferably from 10-60 mg, the active agent being associated with a polymer coating or matrix that comprises a water-insoluble polymer, the polymer coating or matrix providing the dosage form with an extended release of the active agent over at least 12 hours and preferably over at least 16 hours when the dosage form is administered to a patient.

12. The method of claim 11, wherein the active agent cyclobenzaprine is present in the dosage form in an amount of 10-50 mg, preferably 15-45 mg.

13. The method of claim 11, wherein the polymer coating or matrix provides the dosage form with the following dissolution profile when measured in a USP type II apparatus at 50 rpm, at a temperature of 37 degrees C.:
no more than about 40% of the total active agent is released in 2 hours;
from about 40-65% of the total active agent is released after 4 hours;
from about 60-85% of the total active agent is released after 8 hours; and
from about 75-85% of the total active agent is released after 12 hours.

14. The method of claim 11, wherein said water insoluble polymer is selected from ethers of cellulose, esters of cellulose, cellulose acetate, ethyl cellulose, polyvinyl acetate, neutral copolymers based on ethylacrylate and methylmethacrylate, copolymers of acrylic and methacrylic acid esters with quaternary ammonium groups, pH-insensitive ammonio methacrylic acid copolymers, and mixtures thereof.

15. The method of any claim 11, wherein the polymer coating or matrix further comprises a water-insoluble polymer.

16. The method of claim 15, wherein the water soluble polymer is selected from methylcellulose, hydroxypropylcellulose, hydroxypropyl methylcellulose, polyethylene glycol polyvinylpyrrolidone and mixtures thereof.

17. The method of claim 11, wherein the polymer coating or matrix further comprises a plasticizer, in particular selected from triacetin, tributyl citrate, tri-ethyl citrate, acetyl tri-n-butyl citrate, diethyl phthalate, dibutyl sebacate, polyethylene glycol, polypropylene glycol, castor oil, acetylated mono- and di-glycerides and mixtures thereof.

18. The method of claim 11, wherein the dosage form comprises a polymer coating on core particles that comprise the active agent.

19. The method of claim 11, wherein the core particles are formed by the active agent alone or coated on an inert particle core.

20. The method of claim 11 for a long-term treatment of tinnitus and said related auditory dysfunctions extending over 2 weeks or more, preferably 8 weeks or more.

21. A method of treating a subject having been previously diagnosed with and treated for tinnitus, excluding the condition known as “exploding head syndrome” also referred to as “explosive tinnitus”, comprising administering to the subject, via once-a-day oral administration, an effective amount of an extended-release dosage form of cyclobenzaprine as active agent in an amount from 10-80 mg, the active agent being associated with a polymer coating or matrix comprising a water-insoluble polymer, the polymer coating or matrix providing the dosage form with an extended release of the active agent over at least 12 hours and preferably over at least 16 hours when the dosage form is administered to a subject.

22-24. (canceled)
